(19) United States
(12) Patent Application Publication
(10) Pub. No.: US 2017/0319256 A1
(43) Pub. Date: Nov. 9, 2017

(54) METHOD OF SELECTIVELY KILLING CANCER CELLS USING LOW-TEMPERATURE PLASMA JET DEVICE AND METHOD OF TREATING TUMORS USING THE SAME

(71) Applicant: Dong-A University Research Foundation for Industry-Academy Cooperation, Busan (KR)

(72) Inventors: Tae-Hong KANG, Busan (KR); Ji-Ye CHOI, Busan (KR)

(21) Appl. No.: 15/478,558

(22) Filed: Apr. 4, 2017

(30) Foreign Application Priority Data
May 4, 2016 (KR) 10-2016-0055050

(51) Int. Cl. A61B 18/04; A61K 31/502; A61K 31/5365; C12N 5/09

(52) U.S. Cl. CPC A61B 18/042; C12N 5/0693; A61K 31/502; A61K 31/5365; C12N 2500/02

(57) ABSTRACT

A method of selectively killing cancer cells uses a low-temperature plasma jet device. When cancer cells are simultaneously treated with ATR and PARP-1 inhibitors, followed by synchronization of a circadian rhythm and treatment with low-temperature atmospheric-pressure plasma, cancer cell death may be maximized about ten-fold or more compared to when treated with existing low-temperature atmospheric-pressure plasma alone, and thus this method may be usefully used as a future tumor treatment method.

### CROSS-REFERENCE TO RELATED APPLICATIONS

This application claims priority to and the benefit of Korean Patent Application No. 10-2016-0055050, filed on May 4, 2016, the disclosure of which is incorporated herein by reference in its entirety.

### BACKGROUND

### 1. Field of the Invention

The present disclosure relates to a method of selectively killing cancer cells using a low-temperature plasma jet device and a method of treating tumors using the same.

### 2. Discussion of Related Art

Plasma, which is a group of positively charged ions and electrons generated by electric discharge, is drawing attention as a next generation energy source and thus is applied to various industrial fields. For example, plasma is used in mercury lamps, fluorescent lamps, neon signs, semiconductor manufacturing processes, PDPs, ultra-high-temperature nuclear fusion, and the like, and research into application thereof to the biomedical field has recently been conducted.

To have uniform density over a wide area, plasma is generally generated in a pressure-reduced vacuum chamber. In recent times, however, plasma discharging may occur even at a relatively high temperature, i.e., approximately atmospheric pressure, by decreasing a distance between electrodes. Thus, unlike existing low-pressure discharging, a vacuum chamber and a pump device according thereto are not required, and thus costs required for plasma generation are low. In addition, plasma may be used in an atmospheric gas pressure range, and thus has high applicability and may be applied to organic material processing and living bodies.

In particular, research into bio-medical application fields using atmospheric-pressure plasma has started to be conducted, and, at the same time, there has been a growing interest in an effect of non-equilibrium plasma on human cells or tissues. Plasma is known to affect curing of skin damaged by burns or the like, removal and death of malignant cells such as cancer cells, skin improvement for beauty, enamel recovery of bones and teeth, and the like, but any mechanism for inducing the recovery of damaged cells and the death of cancer cells has never been studied. Recently, an atmospheric-pressure low-temperature micro-plasma jet apparatus for bio-medical application or a method of sterilizing a microorganism-contaminated object using atmospheric-pressure plasma has been devised.

In addition, plasma may be divided into high-temperature plasma and low-temperature plasma. When the high-temperature plasma is used medically, it thermally damages cells, and thus glow discharge, which is low-temperature plasma, is used.

Devices using such low-temperature plasma have been developed due to the advantage of not thermally damaging cells or tissues, and many researchers are attempting to apply these devices to cancer treatment studies.

Recently, it has been reported that, when cancer cells such as leukemia, malignant melanoma, and bladder cancer are irradiated with plasma, the death (cell suicide and cell necrosis) of cancer cells increases. In the case of low-temperature atmospheric-pressure plasma, various activated species of plasma may induce necrosis, suicide or the like of cancer cells, thereby killing the cells, but cancer cell killing effects are insignificant. Therefore, there is a need to develop a method of increasing cancer cell killing effects.

### SUMMARY

One or more embodiments provide a method of selectively killing cancer cells using a low-temperature plasma jet device capable of increasing the killing of cancer cells and a method of treating tumors using the same.

According to an aspect of the present invention, there is provided a method of selectively killing cancer cells, including: a first process of treating cancer cells with a kinase inhibitor; a second process of synchronizing a circadian rhythm of normal cells; and a third process of controlling exposure conditions of low-temperature atmospheric-pressure plasma generated by a low-temperature atmospheric-pressure plasma generating apparatus using an alternating current power source when culturing the cancer cells of the first process and the normal cells of the second process.

The kinase inhibitor of the first process may include at least one selected from the group consisting of an ATR inhibitor and a PARP-1 inhibitor.

The kinase inhibitor of the first process may be treated in an amount of 5 μM to 10 μM.

The second process may be performed in a cycle of Zeitgeber time (ZT)17 to ZT22.

The second process may include synchronizing a circadian rhythm with any one selected from the group consisting of mouse embryonic fibroblasts, human fibroblasts, and mouse melanoma cells, which are genetically defective in synchronization.

The exposure conditions may include a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

A distance from a plasma source of the low-temperature atmospheric-pressure plasma generating apparatus to the cancer cells may be set to between 5 cm and 7 cm.

The cancer cells may be exposed to the low-temperature atmospheric-pressure plasma for 15 seconds to 25 seconds.

The cancer cells may be selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.

According to another aspect of the present invention, there is provided a method of treating tumors, including: a first process of administering a kinase inhibitor to an animal with a tumor and synchronizing a circadian rhythm of normal cells; and a second process of treating the animal with low-temperature atmospheric-pressure plasma generated by a low-temperature atmospheric-pressure plasma generating apparatus after the first process.

The kinase inhibitor of the first process may include at least one selected from the group consisting of an ATR inhibitor and a PARP-1 inhibitor.

Exposure conditions of the low-temperature atmospheric-pressure plasma of the second process may include a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

A distance from a plasma source of the low-temperature atmospheric-pressure plasma generating apparatus to the animal may be set to between 5 cm and 7 cm.

The animal may be exposed to the low-temperature atmospheric-pressure plasma for 5 days to 7 days once at 24-hour intervals for 20 seconds to 30 seconds every time.

The tumors may be selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A and 1B illustrate images and graphs showing results of confirming a cell death-enhancing effect according to addition of oxygen gas while human cancer cells are treated with non-thermal plasma (NTP), according to an embodiment of the present disclosure;

FIGS. 2A to 2C illustrate images and graphs showing NTP and NTP combined with oxygen gas (NTPO)-induced genomic DNA lesions and breaks, according to an embodiment of the present disclosure;

FIGS. 3A and 3B illustrate images and graphs showing activation of the ATR-CHK1 pathway in response to NTP-induced DNA damage response, according to an embodiment of the present disclosure;

FIGS. 4A and 4B illustrate images and graphs showing reinforced DNA breaks in plasma treatment with a PARP inhibitor, according to an embodiment of the present disclosure;

FIGS. 5A to 5C illustrate images and graphs showing PARP-1 inhibition augments apoptosis during NTP and NTPO treatment, according to an embodiment of the present disclosure;

FIGS. 6A to 6D illustrate images and graphs showing circadian oscillation of PARP-1 activity in normal fibroblasts, according to an embodiment of the present disclosure; and

FIGS. 7A and 7B illustrate images and graphs showing PARP-1 activity dictates circadian toxicity of NTP and NTPO in normal cells, according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Hereinafter, various aspects and embodiments of the present disclosure will be described in more detail.

The present disclosure provides a method of selectively killing cancer cells, including the following processes: a first process of treating cancer cells with a kinase inhibitor; a second process of synchronizing a circadian rhythm of normal cells; and a third process of controlling exposure conditions of low-temperature atmospheric-pressure plasma (non-thermal plasma (NTP) formed at atmospheric pressure) generated by a low-temperature atmospheric-pressure plasma generating apparatus using an alternating current power source when the cancer cells of process 1 and the normal cells of process 2 are cultured.

According to the method of selectively killing cancer cells as described above, it was verified through experimental examples that a cancer cell killing effect increased about six-fold compared to existing double-stranded DNA cleavage, radiation therapy mainly induced by efficiently generating a high mutation rate, and existing treatment with low-temperature atmospheric-pressure plasma alone.

In the first process, the kinase inhibitor may include at least one selected from the group consisting of an ataxia telangiectasia mutated (ATM) and Rad3-related (ATR) inhibitor and a poly [ADP-ribose]polymerase 1 (PARP-1) inhibitor.

As an exemplary example, the ATR inhibitor and the PARP-1 inhibitor may be administered in combination.

Any material may be used as the ATR inhibitor without limitation so long as it is capable of inhibiting ATR. For example, the ATR inhibitor may be VE-821, VE-822, and ETP-46464, but the present disclosure is not limited thereto.

Any material may be used as the PARP-1 inhibitor without limitation so long as it is capable of inhibiting PARP-1. For example, the PARP-1 inhibitor may be olaparib (AZD2281), but the present disclosure is not limited thereto.

In the method of selectively killing cancer cells, according to the present disclosure, ATR-mediated cell cycle checkpoints and PARP-1-dependent DNA recovery may be induced by administering the ATR inhibitor and the PARP-1 inhibitor together, which target the ATR and PARP-1 pathways.

The kinase inhibitor of the first process may be treated in an amount of 5 μM to 10 μM.

In addition, in the second process of synchronizing a circadian rhythm of normal cells, the synchronization of the circadian rhythm may be performed in a cycle of ZT17 to ZT22, and may include synchronizing a circadian rhythm with any one selected from mouse embryonic fibroblasts (Cry1/2 knockout and Perl/2 knockout), human fibroblasts, mouse melanoma cells, and the like, which are genetically defective in synchronization, but the present disclosure is not limited thereto.

Meanwhile, a circadian timing (circadian rhythm) system consists of a molecular clock which induces 24-hour changes in nearly all cytophysiological processes including cell cycles, DNA recovery, and cell death. Chronotherapeutics, which generally apply such a circadian timing system to treatment, aim to decrease resistance to drugs and/or to enhance efficiency of drugs through treatment and management according to biorhythms. Despite this cumulative data, however, there are no generally used parameters in clinical trials that may affect timing, efficacy and associated side effects of cancer treatment.

However, in the present disclosure, it is confirmed that an effect of significantly decreasing cell survival rates according to DNA damage response is obtained by administering the PARP-1 inhibitor before the synchronization of the circadian rhythm and inducing periodic activity with NTPO.

The method may further include transfecting the cancer cells with double-stranded siRNA before the synchronization of the circadian rhythm.

The siRNA of the cancer cells is basically a complete form in which two strands of RNA are paired to form a double strand, and siRNA may be directly synthesized in vitro and then introduced into a cell through transfection, or may be a modified form with a short hairpin so that it can be used for transfection by plasmid-based shRNA vectors, PCR-derived siRNA expression cassettes, and the like.

siRNA may be synthesized by various methods known in the art, such as a method of directly synthesizing siRNA chemically (Sui G et al., Proc Natl Acad Sci USA, 99:5515-5520, 2002), a synthesis method using in vitro transcription (Brummelkamp TR et al., Science, 296:550-553, 2002), a method of cleaving long double-stranded RNA synthesized by in vitro transcription, with an RNaseIII family enzyme (Paul C P et al., Nature Biotechnology, 20:505-508, 2002), and the like.

The low-temperature atmospheric-pressure plasma described in the present specification has high chemical reactivity to a target object without thermal changes, has relatively stable energy, and acts only on a surface of a reactant, and thus does not change states of interacting materials nor damage interacting materials.

Exposure conditions of the low-temperature atmospheric-pressure plasma generating apparatus used in the method of selectively killing cancer cells may include a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

In addition, a distance from a plasma source of the low-temperature atmospheric-pressure plasma generating apparatus to the cancer cells may be set to between 5 cm and 7 cm.

Plasma exposure conditions refer to the number of times and time of exposure to plasma, and the cancer cells may be exposed to low-temperature atmospheric-pressure plasma for 5 days to 7 days once at 24-hour intervals for 20 seconds or 30 seconds every time.

Comprehensively, it is confirmed that, according to the present disclosure, the synchronization of the circadian rhythm is performed, and oxygen is supplied while treating with low-temperature atmospheric-pressure plasma and, accordingly, a cancer cell killing effect is about 2.5 times greater than that in a case in which the synchronization of the circadian rhythm is not performed. Thus, the death of cancer cells may be maximized and thus this method may be usefully used in cancer treatment.

In the present disclosure, the method of selectively killing cancer cells is performed using a low-temperature atmospheric-pressure plasma generating apparatus, and thus may be readily applied to superficial cancer such as skin cancer and breast cancer and may also be applied to treatment of nearly all types of cancer including internal cancer.

The cancer cells may be, for example, one selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.

As described above, according to the method of selectively killing cancer cells, it is verified through experimental examples that the cancer cell killing effect increases about six-fold compared to existing double-stranded DNA cleavage, radiation therapy mainly induced by efficiently generating a high mutation rate, and existing treatment with low-temperature atmospheric-pressure plasma alone. Thus, the present disclosure may be applied to a method of treating tumors, including: a first process of administering a kinase inhibitor to an animal with a tumor, transfecting tumor cells with double-stranded siRNA, and then synchronizing a circadian rhythm; and a second process of treating the animal with low-temperature atmospheric-pressure plasma generated by a low-temperature atmospheric-pressure plasma generating apparatus.

In the present disclosure, the kinase inhibitor of the first process may include at least one selected from the group consisting of an ATR inhibitor and a PARP-1 inhibitor.

The ATR inhibitor and the PARP-1 inhibitor may be simultaneously used.

In the present disclosure, exposure conditions of the low-temperature atmospheric-pressure plasma of the second process may include a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

A distance from a plasma source of the low-temperature atmospheric-pressure plasma generating apparatus to the animal may be set to between 5 cm and 7 cm.

In addition, the animal may be exposed to low-temperature atmospheric-pressure plasma for 5 days to 7 days once at 24-hour intervals for 20 seconds or 30 seconds every time.

In the present disclosure, the tumor may be one selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.

Hereinafter, the present disclosure will be described in further detail with reference to the following examples, but these examples are not to be construed as limiting the scope and content of the present disclosure. In addition, based on the disclosure of the invention including the following examples, it is obvious that the present disclosure in which experimental results are not specifically shown may be easily carried out by one of ordinary skill in the art, and these modifications and changes are within the appended claims.

### Example 1

(1) Process 1: Plasma Treatment

Cells were treated using a previously reported plasma jet device. A typical operating condition of the pulsed-dc plasma jet has the applied voltage 1.6 kV, repetition frequency 50 kHz, and duty ratio 10%. The working gas (helium) and reactive gas (oxygen) flow rate were kept constant at 500 and 5 SCCM (SCCM denotes cubic centimeter per minute at standard temperature and pressure), respectively. The cells were cultured on 12-mm microscope cover glass coated with gelatin from porcine skin (Sigma-Aldrich) and were treated with helium generated-NTP with or without the flow of oxygen gas. The exposed cover glass was then transferred to 12-well plates containing fresh medium and incubated at 37° C. in a humidified incubator supplemented with 5% CO2. If necessary, the cells were treated with 5 or 10 μM of specific inhibitors targeting ATR (ETP46464), ATM (KU55933), DNA-PK (NU7026), or PARP1 (AZD2281) for 1 h before plasma treatment and kept until cell harvest. The inhibitors were purchased from Selleck Chemicals.

(2) Process 2: Cell Culture, siRNA Transfection, and Synchronization of Circadian Rhythm

Wild-type mouse embryonic fibroblasts (WT-MEF) and CRY1 and CRY2 double knockout mouse embryonic fibroblasts (CRY^{DKO} MEF, a gift from Dr. KJ Kim, Seoul National University) were cultured in Dulbecco's Modified Eagle's Medium (Hyclone) supplemented with 10% fetal bovine serum (Hyclone) and 1% penicillin-streptomycin (Hyclone). Human lung carcinoma A549 and melanoma SK-MEL2 cells were cultured in Dulbecco's Modified Eagle's Medium supplemented with 10% fetal bovine serum and 1% penicillin-streptomycin and in Eagle's Minimum Essential Medium (Hyclone) supplemented with 10% fetal bovine serum, 1% penicillin-streptomycin, 1% sodium pyruvate (Gibco), and 1% non-essential amino acids (Gibco), respectively. If necessary, the cells were transfected with siRNA duplexes targeting XPA (Dharmacon) using Lipofectamine® 2000 (Invitrogen) according to the manufacturer's protocol. Plasma was treated after incubation for 48 h. For circadian synchronizations, MEF cells were treated and cultured as previously reported

### Experimental Example 1: Immunofluorescence

For immunofluorescence staining, cells were fixed with 4% formaldehyde (Sigma-Aldrich) for 10 min at room temperature and permeabilized with 0.5% Triton™ X-100 (Bio Basic). Specific antibodies against 8-OxoG (Abcam), phospho-histone H2AX (Ser139; Millipore), phospho-CHK1 (Ser345), phospho-P53 (Ser15), PARP1, cleaved-caspase 3 (Cell Signaling Technology), and poly-ADP-ribose (Enzo Life Sciences) were used for visualization of the proteins. The images were captured using a fluorescence microscope (Nikon) equipped with the NIS-Elements 4.0 Nikon imaging software. For quantification, a minimum of 500 cells were analyzed from each of three independent experiments.

### Experimental Example 2: Immunoblotting

Harvested cells were resuspended in 100 μl of 1× lysis buffer (20 mM Tris-HCl pH 6.8, 150 mM NaCl, 1 mM EDTA, 1 mM EGTA, protease inhibitor cocktail, and 10% Triton™ X-100) and sonicated using a sonicator (SONICS). Total protein (30 μg) was run on 10% SDSpolyacrylamide gels and transferred to nitrocellulose blotting membranes using electrophoresis chambers (Bio-RAD Laboratories). Membranes were analyzed by immunoblotting with antibodies for XPA (Kamiya Biomedical Company), CRY1, BMAL1 (Santa Cruz Biotech), and GAPDH (Cell Signaling Technology).

### Experimental Example 3: Gene Comet Assay

DNA breaks were determined using the CometAssay® Kit (Trevigen). Briefly, after 24 h of plasma treatment, the cells were mixed at a 1:10 (v/v) ratio with low-melting-point agarose at 37° C. The cell suspension (75 μl) was dispersed onto a microscope comet slide (MF) and maintained at 4° C. for 40 min. The cells were then lysed at 4° C. and incubated in alkaline solution with 200 mM NaOH and 1 mM EDTA for 40 min at room temperature to allow DNA unwinding. The slides were then placed and run in a horizontal electrophoresis system. Afterward, the slides were gently immersed twice in dH₂O for 5 min each and in 70% ethanol for 5 min, and then dried overnight at room temperature. Before comet scoring, the DNA was counterstained with SYBR® green for visualization under a fluorescence microscope. The tail moment was calculated using Comet Assay Score software Project (CASP) software.

### Experimental Example 4: Terminal Deoxynucleotide Transferase dUTP Nick Terminal Labeling (TUNEL) Analysis

For the detection of DNA fragmentation, the Click-iT® TUNEL Alexa Fluor® Imaging Assay (Invitrogen) was used according to the manufacturer's instructions. Briefly, cells were fixed in 4% formaldehyde for 10 min at room temperature and permeabilized in 0.5% Triton™ X-100 in PBS for 20 min at room temperature. The cells were then incubated for 60 min at 37° C. in terminal deoxynucleotidyl transferase enzyme reaction mixture. The cells were washed twice with 1×PBST for 2 min each and then incubated for 30 min at room temperature with Click-iT® reaction mixture. Cell nuclei were counterstained with Hoechst 33342 (Sigma-Aldrich) for 15 min at room temperature and images were taken under a fluorescence microscope.

Statistics

Statistical significance was determined using Student's t-test. Bars and error bars were presented as mean±SD from at least three independent experiments. Differences were considered significant at the values of p<0.05 (*), p<0.01 (**), and p<0.001 (***). All statistical analyses were performed using the GraphPad Prism 5.0 software (GraphPad).

Results

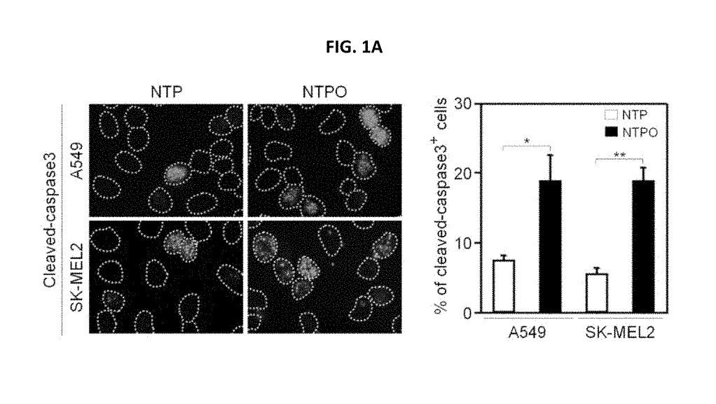

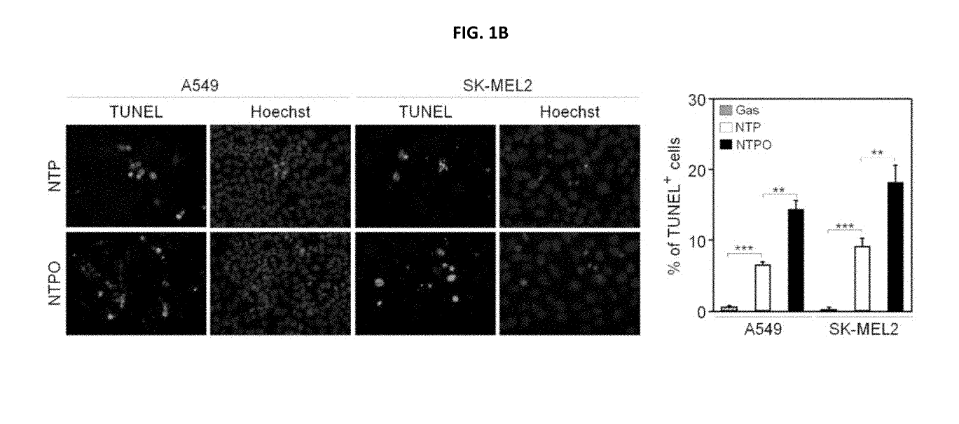

As shown in FIG. 1A, it was confirmed that, when treated with NTPO, cleaved-caspase 3 was activated three-fold more in A549 and SK-MEL2 (melanoma) human cancer cells than in a case of NTP treatment, and, As shown in FIG. 1B, it was confirmed that TUNEL-positive cells were produced two-fold more than the case of NTP treatment. These results indicate that the cell death may be increased by injection of oxygen gas during NTP treatment.

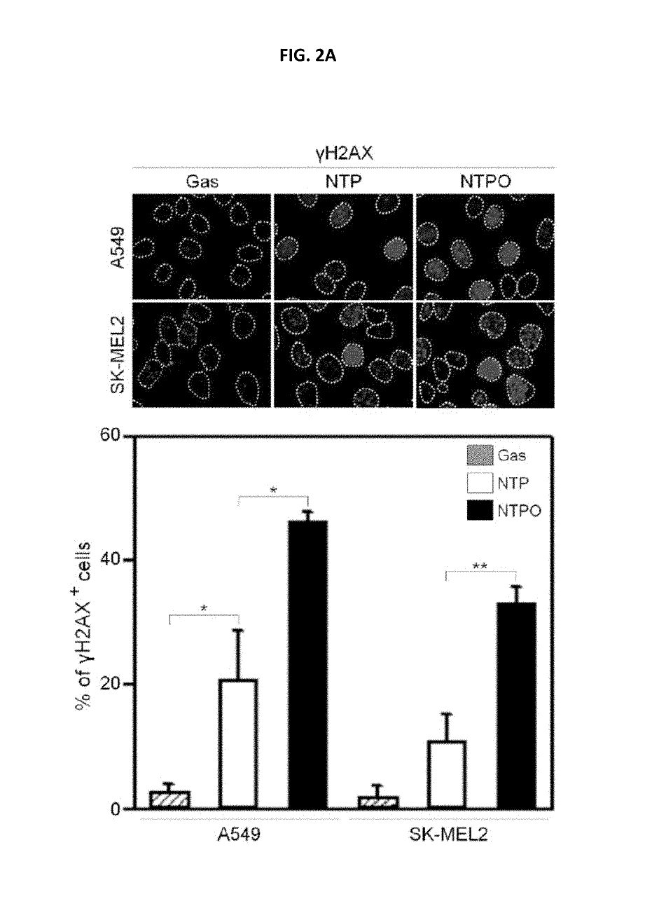

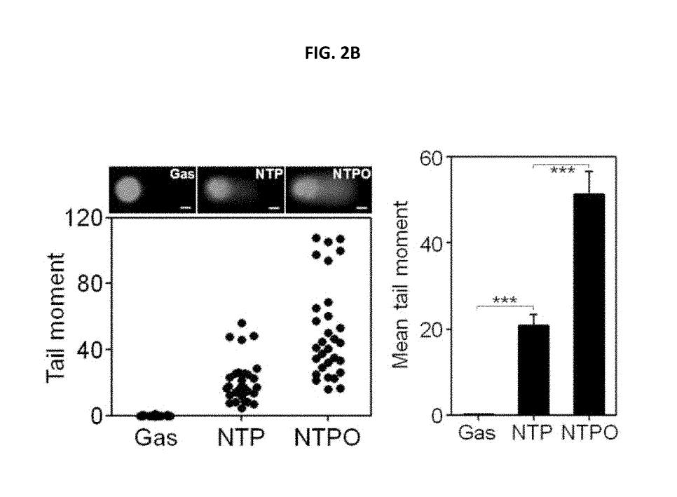

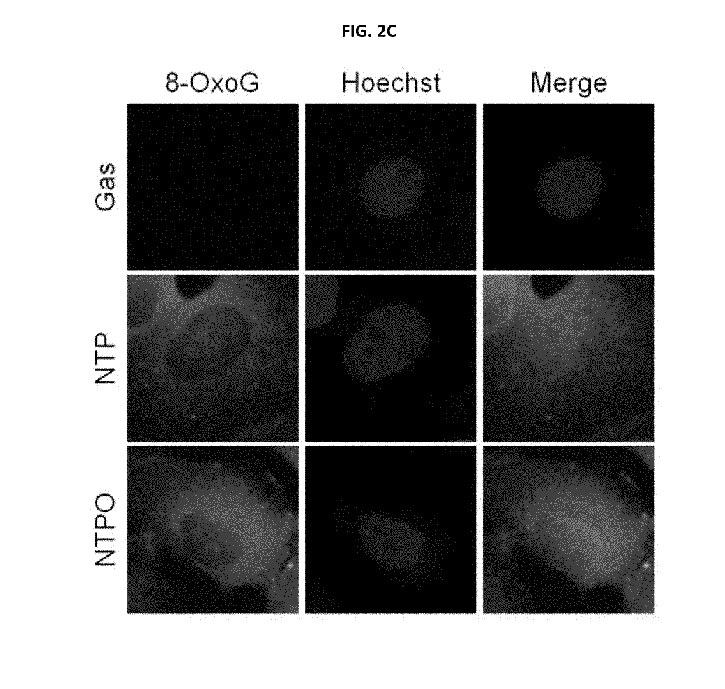

In addition, As shown in FIG. 2A, in both A549 and SK-MEL2 cells, the phosphorylation of a variant histone H2AX (γH2AX), a general marker for DNA breaks, appeared after 2 h in both NTP- and NTPO-exposed cells, whereas cells exposed to a gas (helium) control exhibited no significant γH2AX phosphorylation.

In addition, using a comet assay, we detected the so-called ‘comet-shaped nuclei’ as a result of DNA breaks in the NTP- and NTPO-treated cells (FIG. 2B). Further, the quantitative analysis indicated that NTPO produced approximately 2-fold stronger γH2AX phosphorylation and 3-fold more comet nuclei than NTP (FIGS. 1A and 1B).

Furthermore, as shown in FIG. 2C, it was confirmed that cytoplasm as well as nuclei of 8 OxoG cells were stained in the cases of NTP treatment and NTPO treatment.

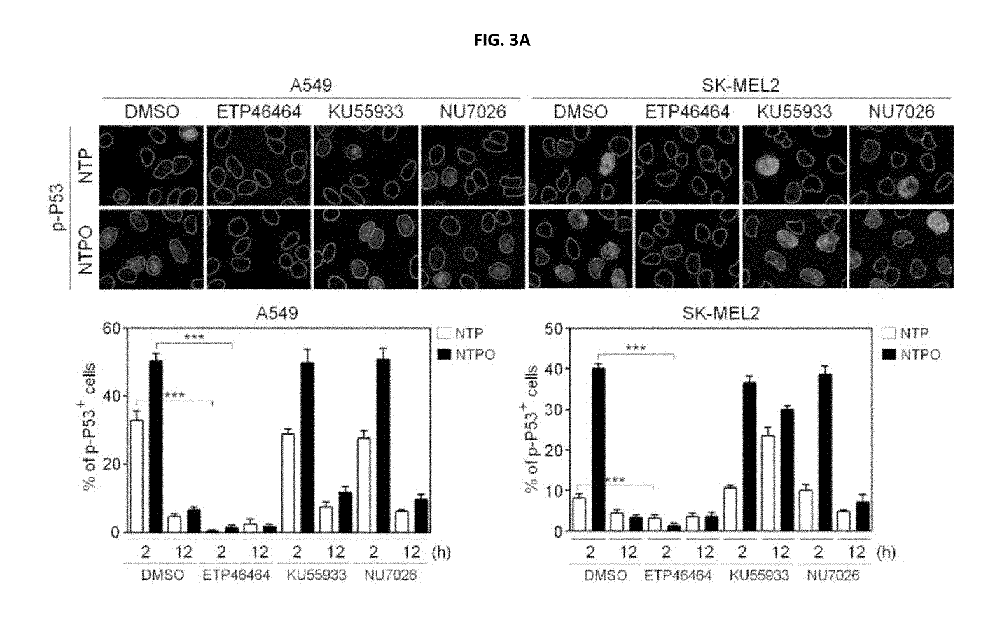

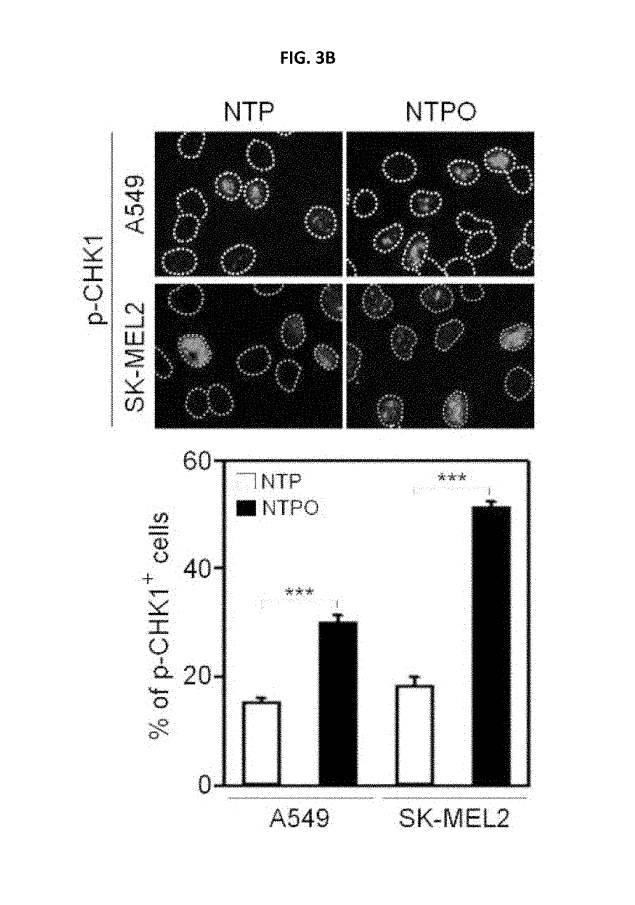

Meanwhile, ATR, ATM and DNA-PK kinases, which are provided as pre-initial sensors to cells in mammals, are synchronized with the cell cycle to secure time for DNA repair in response to genotoxic stress. As a result of pretreating A549 and SKMEL2 cells with an inhibitor specific to ATR (ETP 46464), ATM (KU55933), or DNA-PK (NU7026), followed by treatment with NTP or NTPO, phosphorylation was not shown in the pretreatment with ATR (ETP 46464) As shown in FIG. 3A, and CHK1 phosphorylation was confirmed as a result of flowing oxygen gas while treating with NTP.

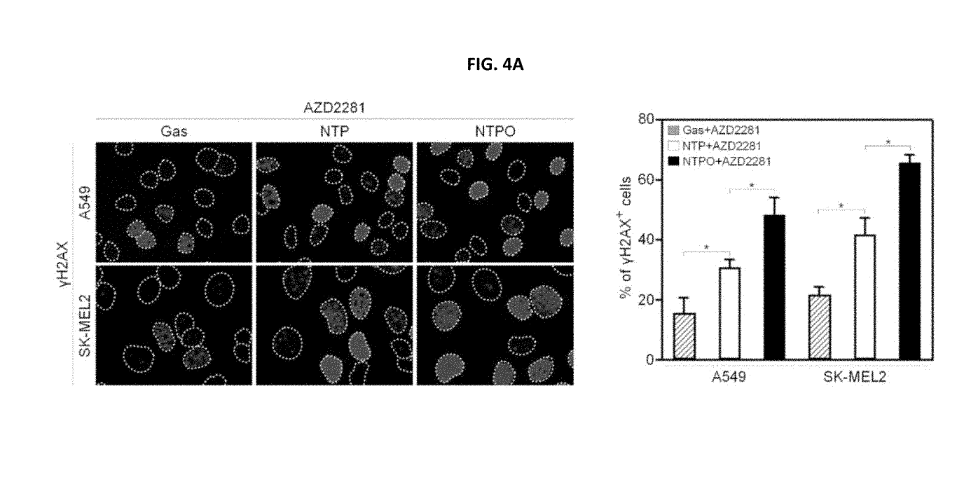

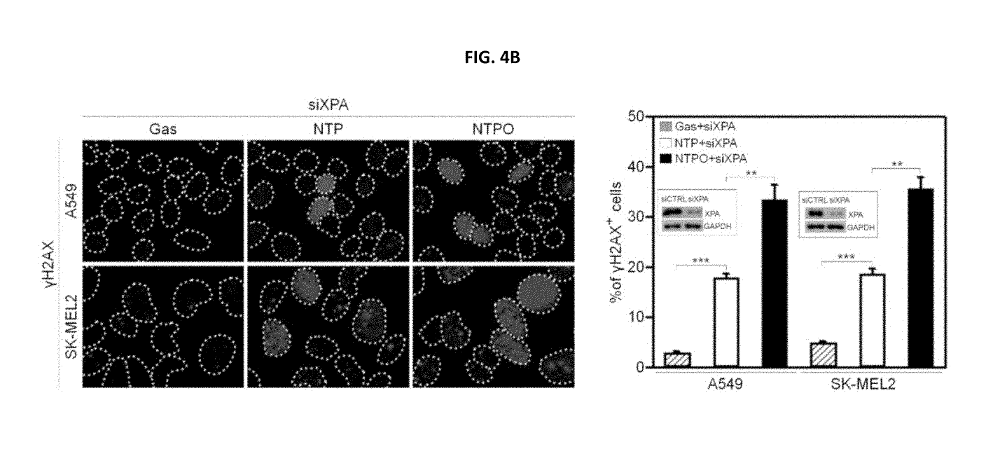

To search for a method of enhancing NTP treatment efficiency based on these results, in the present disclosure, both the A549 cells and the SK-MEL2 cells were treated with an inhibitor (AZD2281) selective to PARP-1 and, as a result, it was confirmed that γH2AX phosphorylation was significantly increased by treatment of NTP or NTPO (see FIG. 4A). In particular, the phosphorylation of γH2AX, which is not generally recognized well, was measured even in the case of gas control, while, as a result of blocking the NER pathway by knockdown XPA, which is a key factor for the NER mechanism, distinct changes in γH2AX phosphorylation were not detected in the case of treatment with NTP or NTPO (see FIG. 4B).

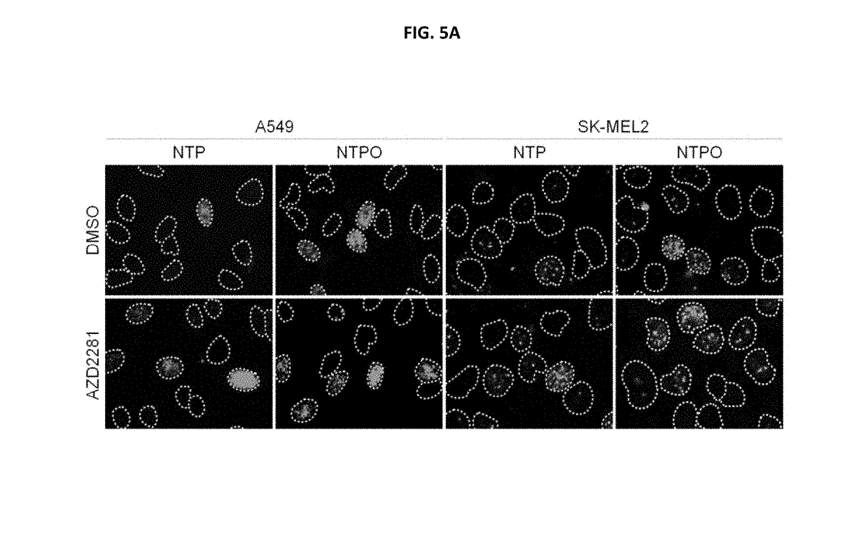

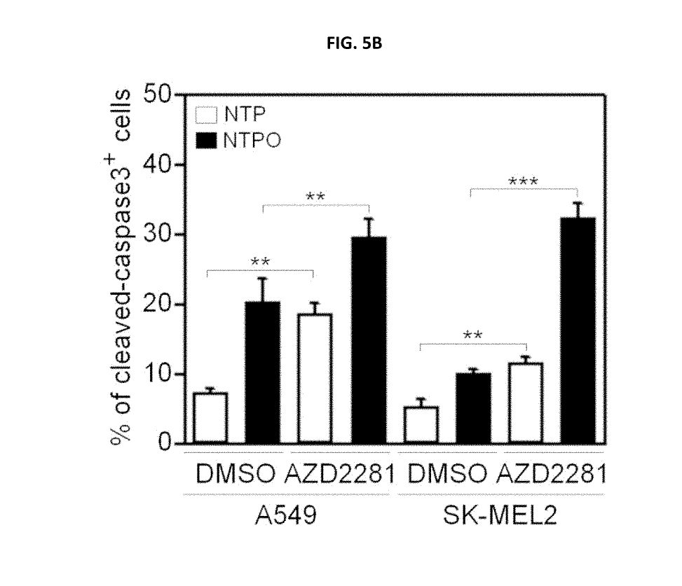

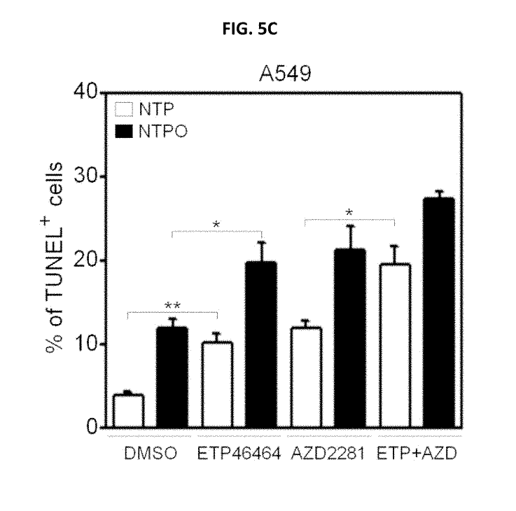

From results of cleaved-caspase 3 staining (see FIG. 5A) and TUNEL activity measurement (see FIG. 5B) 24 hours after treatment with NTP or NTPO, pharmacological inhibition of PARP-1 activity was confirmed as a cell death signal increased.

In addition, As shown in FIG. 5A, NTP efficiency generally coincides with NTPO and a cell killing effect in the presence of an ATR inhibitor or a PARP-1 inhibitor, and is further increased by addition of the ATR or PARP-1 inhibitor, As shown in FIG. 5B.

Importantly, as can be confirmed in FIG. 5C, when the ATP and PARP-1 inhibitors were simultaneously administered, an important synergistic effect was detected when inhibiting NTP-induced cells, but the effect was insignificant during NTPO-induced cell death.

These results show that the PARP-1 activity has a circadian rhythm in normal mouse cells.

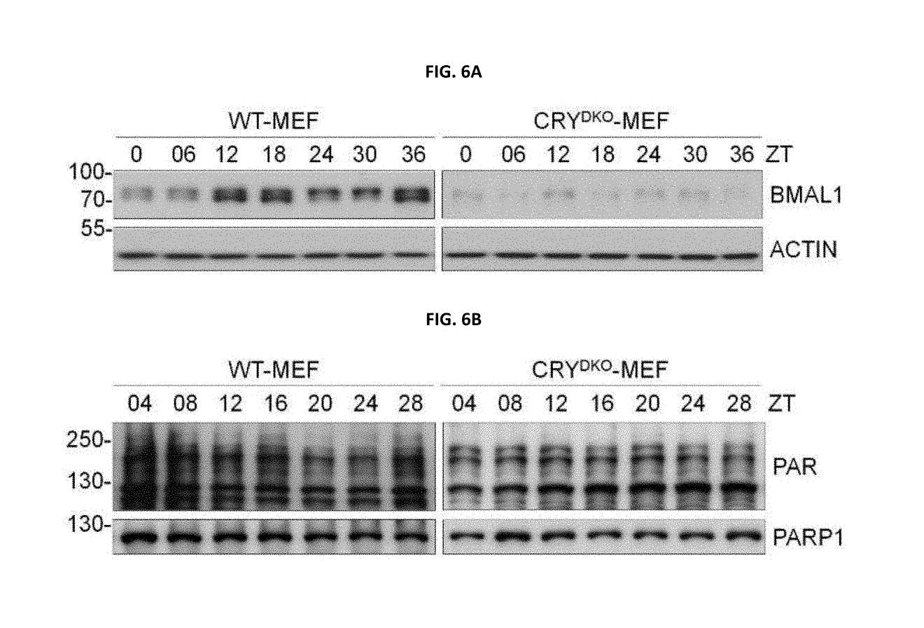

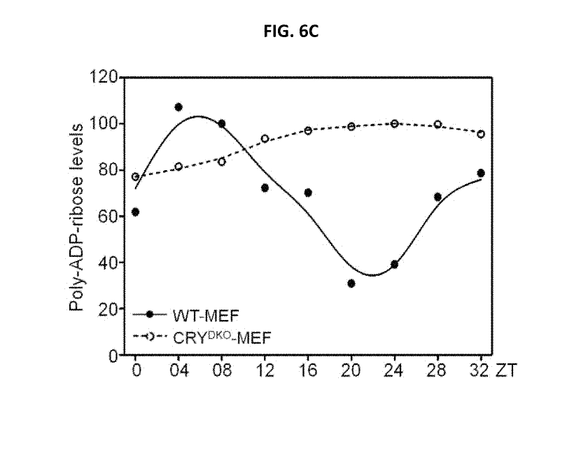

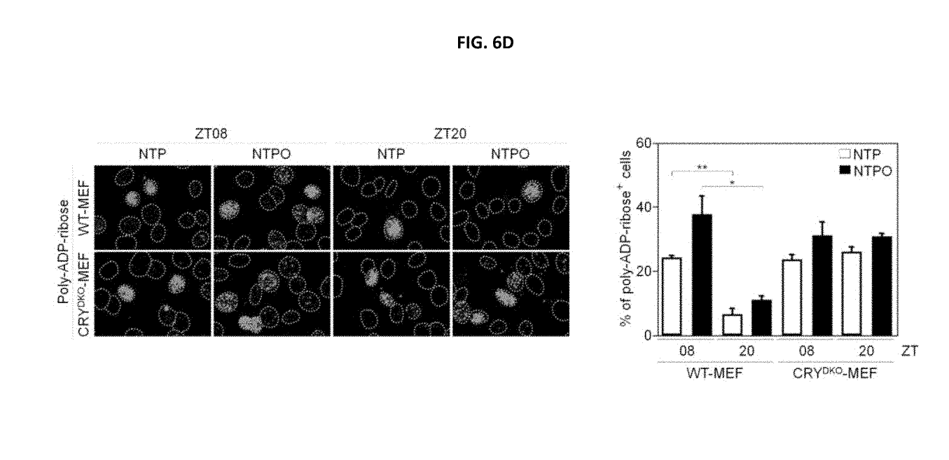

To examine the role of PARP-1 in normal cells having NTP- or NTPO-induced genotoxicity, mouse embryonic fibroblasts having an active circadian clock (WT-MEF) and mouse embryonic fibroblasts having an inactive clock due to the loss of central clock elements of cytochrome 1 and 2 (CRY^{DKO}-MEF) were used. As shown in FIG. 6A, forskolin treatment generated a robust circadian oscillation of a clock-controlled gene BMAL1 as a readout for clock activity in WT-MEF, but not in CRYDKO-MEF.

Importantly, as shown in FIG. 6B, PARP1 activity deduced from total PAR levels showed a circadian rhythm in WT-MEF but not in CRYDKO-MEF, whereas PARP1 protein levels remained unchanged.

In addition, to examine an effect of clock activity on the PARP-1 activity when treating with plasma, cells were treated with NTP or NTPO at ZT08 (ZT0: Zeitgeber 0 hour in treatment with forskolin) and ZT20 which represent a maximum PAR signal and a minimum PAR signal, respectively. As a result, as shown in FIG. 6C, synchronization of a circadian rhythm was confirmed in the case of induction with forskolin. Furthermore, as shown in FIG. 6D, it was confirmed that, in the case of the WT-MEF, the number of PAR-positive cells was five times greater at ZT08 than at ZT20, while, after treatment with NTP or NTPO, differential circadian PARP-1 activity was not detected in the CRY^{DKO}-MEF, and the number of PAR-positive cells was similar in the two cases regardless of ZT.

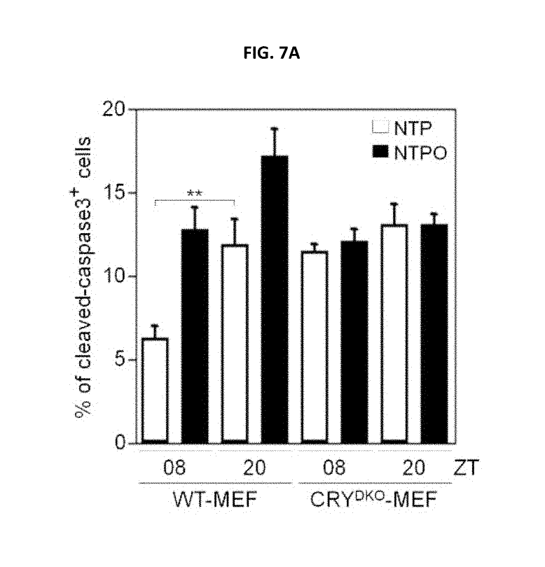

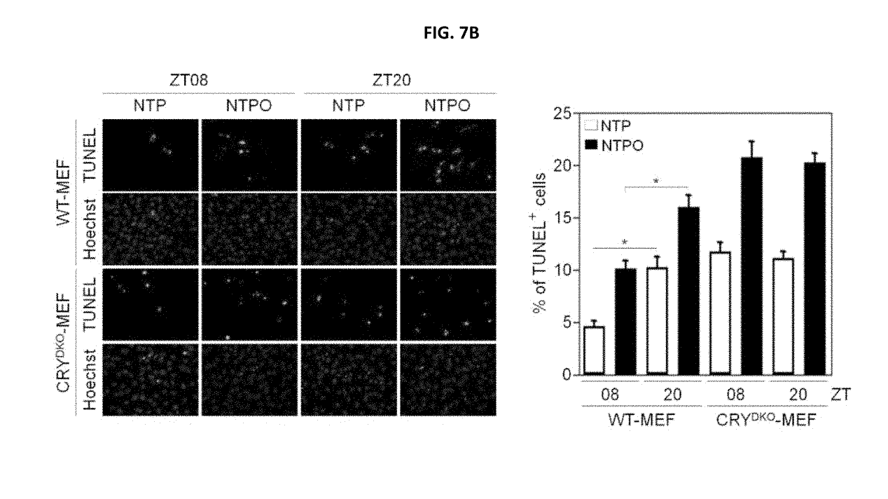

Thus, as shown in FIGS. 7A and 7B, in cell viability according to treatment with NTP or NTPO, it was confirmed that the WT-MET case exhibited a smaller number of death cells at ZT08 than at ZT20, while the number of death cells in the CRY^{DKO}-MEF case was similar at ZT08 and ZT20 to that in the case of cells treated with NTP or NTPO.

These results mean that a circadian rhythm of a patient needs to be considered in treatment with NTP or NTPO to minimize side effects of damage to normal cells.

Taken together with all the results, in cancer cells, it is verified that NTP- or NTPO-induced DNA damage is cured by activating ATR-mediated cell cycle checkpoints and PARP-1-dependent recovery pathways, and thus, to enhance cancer treatment efficiency using NTP or NTPO, an ATR inhibitor and a PARP-1 inhibitor may be treated alone or simultaneously treated. In addition, to minimize damage to normal cells positioned in the vicinity of cancer cells, which may occur in treatment, a circadian rhythm of PARP-1 is perceived as being present in normal cells, and cancer treatment may be performed at Zeitgeber time (ZT) having a high activity of PARP-1 and, to accordingly, such cancer treatment may be applied to plasma-chronochemotherapy capable of minimizing the loss of normal cells.

According to the present disclosure, when cancer cells are treated with an ATR inhibitor or a PARP-1 inhibitor, followed by synchronization of a circadian rhythm and low-temperature atmospheric-pressure plasma treatment, the death of the cancer cells may be maximized with a very high yield compared to when treated with existing low-temperature atmospheric-pressure plasma alone, and thus this method may be usefully used as a future tumor treatment method.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A method of selectively killing cancer cells, the method comprising:
a first process of treating cancer cells with a kinase inhibitor;
a second process of synchronizing a circadian rhythm of normal cells; and
a third process of controlling exposure conditions of low-temperature atmospheric-pressure plasma generated by a low-temperature atmospheric-pressure plasma generating apparatus using an alternating current power source when the cancer cells of the first process and the normal cells of the second process are cultured.

2. The method of claim 1, wherein the kinase inhibitor of the first process comprises at least one selected from the group consisting of an ATR inhibitor and a PARP-1 inhibitor.

3. The method of claim 1, wherein the kinase inhibitor of the first process is treated in an amount of 5 μM to 10 μM.

4. The method of claim 1, wherein the second process is performed in a cycle of Zeitgeber time (ZT)17 to ZT22.

5. The method of claim 1, wherein the second process comprises synchronizing a circadian rhythm with any one selected from the group consisting of mouse embryonic fibroblasts, human fibroblasts, and mouse melanoma cells, which are genetically defective in synchronization.

6. The method of claim 1, wherein the exposure conditions comprise a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

7. The method of claim 1, wherein a distance from a plasma source of the low-temperature atmospheric-pressure plasma generating apparatus to the cancer cells is set to between 5 cm and 7 cm.

8. The method of claim 1, wherein the cancer cells are exposed to the low-temperature atmospheric-pressure plasma for 15 seconds to 25 seconds.

9. The method of claim 1, wherein the cancer cells are selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.

10. A method of treating tumors, the method comprising:
a first process of administering a kinase inhibitor to an animal with a tumor and synchronizing a circadian rhythm of normal cells; and
a second process of treating the animal with low-temperature atmospheric-pressure plasma generated by a low-temperature atmospheric-pressure plasma generating apparatus after the first process.

11. The method of claim 10, wherein the kinase inhibitor of the first process comprises at least one selected from the group consisting of an ATR inhibitor and a PARP-1 inhibitor.

12. The method of claim 10, wherein exposure conditions of the low-temperature atmospheric-pressure plasma of the second process comprise a helium gas flow of 400 sccm to 600 sccm, an oxygen gas flow of 3 sccm to 6 sccm, an applied voltage of 1 kV to 2 kV, 40 kHz to 60 kHz, and a duty ratio of 8% to 12%.

13. The method of claim 10, wherein a distance from a plasma source of the low-temperature atmospheric-pressure plasma generating apparatus to the animal is set to between 5 cm and 7 cm.

14. The method of claim 10, wherein the animal is exposed to the low-temperature atmospheric-pressure plasma for 5 days to 7 days once at 24-hour intervals for 20 seconds to 30 seconds every time.

15. The method of claim 10, wherein the tumors are selected from the group consisting of skin cancer, carcinoma, lymphoma, blastoma, sarcoma, liposarcoma, neuroendocrine tumors, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, brain cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney and renal cancer, prostate cancer, vulvar cancer, thyroid cancer, hepatic carcinoma, anal carcinoma, penile carcinoma, testicular cancer, esophageal cancer, biliary tract cancer, and head and neck cancer.
